(19) United States
(12) Patent Application Publication
(10) Pub. No.: US 2010/0076299 A1
(43) Pub. Date: Mar. 25, 2010

(54) Inducing Desirable Temperature Effects On Body Tissue Using Alternate Energy Sources

(75) Inventors: Rolfe Tyson Gustus, San Diego, CA (US); Corbett W. Stone, San Diego, CA (US); Michael F. Hoey, Shoreview, MN (US); Arthur G. Blanck, Ramona, CA (US); Len Briggs, Carlsbad, CA (US); Mike Perry, Los Altos, CA (US); Meital Mazor, San Diego, CA (US); Linas R. Kunstmanas, Valley Center, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Minnow Medical, Inc., San Diego, CA (US)

(21) Appl. No.: 12/564,268

(22) Filed: Sep. 22, 2009

Related U.S. Application Data
(60) Provisional application No. 61/099,155, filed on Sep. 22, 2008.

(51) Int. Cl. A61N 7/02; A61B 18/20; A61B 18/24; A61B 18/18; A61B 6/00; A61B 5/055

(52) U.S. Cl. 600/411; 606/14; 606/15; 601/3; 606/33; 600/478

(57) ABSTRACT

A system for inducing desirable temperature effects on body tissue, the body tissue being disposed about a lumen. The system includes an elongate catheter having a proximal end and a distal end with an axis therebetween with an energy delivery portion for transmission of energy. A tissue analyzer configured to characterize the body tissue in the lumen proximate the energy delivery portion and an energy source coupled to the energy delivery portion transmitting tissue treatment energy, wherein the energy is non-RF energy. A processor coupled to the tissue analyzer and energy source, the processor configured to determine an appropriate treatment energy for the characterized body tissue so as to mildly heat the body tissue with the energy delivery portion without ablating.

### CROSS-REFERENCES TO RELATED APPLICATIONS

The present application claims the benefit under 35 USC 119(e) of U.S. Provisional Application No. 61/099,155 filed Sep. 22, 2008; the full disclosure of which is incorporated herein by reference in its entirety.

This application is related to U.S. patent application Ser. No. 11/975,474, filed on Oct. 18, 2007, entitled “Inducing Desirable Temperature Effects on Body Tissue”; U.S. patent application Ser. No. 11/975,383, filed on Oct. 18, 2007, and entitled “System For Inducing Desirable Temperature Effects On Body Tissue”; and U.S. patent application Ser. No. 11/122,263, filed on May 3, 2005, entitled “Imaging and Eccentric Atherosclerotic Material Laser remodeling and/or Ablation Catheter”, the full disclosures of which are incorporated herein by reference.

### STATEMENT AS TO RIGHTS TO INVENTIONS MADE UNDER FEDERALLY SPONSORED RESEARCH AND DEVELOPMENT

NOT APPLICABLE

### BACKGROUND OF THE INVENTION

The present invention is generally related to medical devices, systems, and methods. In exemplary embodiments, the invention provides treatment for luminal diseases, particularly for atherosclerotic plaque, vulnerable or “hot” plaque, and the like. The structures of the invention allow remodeling artery tissue using gentle heat without ablation.

Balloon angioplasty and other catheters often are used to open arteries that have been narrowed due to atherosclerotic disease. The trauma associated with balloon dilation can impose significant injury, so that the benefits of balloon dilation may be limited in time. Stents are commonly used to extend the beneficial opening of the blood vessel. Restenosis or a subsequent narrowing of the body lumen after stenting has occurred in a significant number of cases.

More recently, drug coated stents (such as Johnson and Johnson's Cypher™ stent, the associated drug comprising Sirolimus™) have demonstrated a markedly reduced restenosis rate, and others are developing and commercializing alternative drug eluting stents. In addition, work has also been initiated with systemic drug delivery (intravenous or oral) which may also improve the procedural angioplasty success rates.

While drug eluting stents appear to offer significant promise for treatment of atherosclerosis in many patients, there remain many cases where stents either cannot be used or present significant disadvantages. Generally, stenting leaves an implant in the body. Such implants can present risks, including mechanical fatigue, corrosion, and the like, particularly when removal of the implant is difficult and involves invasive surgery. Stenting may have additional disadvantages for treating diffuse artery disease, for treating bifurcations, for treating areas of the body susceptible to crush, and for treating arteries subject to torsion, elongation, and shortening.

A variety of modified restenosis treatments or restenosis-inhibiting treatment modalities have also been proposed, including intravascular radiation, cryogenic treatments, ultrasound energy, and the like, often in combination with balloon angioplasty and/or stenting. While these and different approaches show varying degrees of promise for decreasing the subsequent degradation in blood flow following angioplasty and stenting, the trauma initially imposed on the tissues by angioplasty remains problematic.

A number of alternatives to stenting and balloon angioplasty so as to open stenosed arteries have also been proposed. For example, a wide variety of atherectomy devices and techniques have been disclosed and attempted. Despite the disadvantages and limitations of angioplasty and stenting, atherectomy has not gained the widespread use and success rates of dilation-based approaches. More recently, still further disadvantages of dilation have come to light. These include the existence of vulnerable plaque, which can rupture and release materials that may cause myocardial infarction or heart attack.

In light of the above, it would be advantageous to provide methods and systems for inducing vasodilation in artery tissue and remodeling of the lumens of the body. It would further be desirable to avoid significant cost or complexity while providing structures which could remodel body lumens without having to resort to the trauma of extreme dilation, and to allow the opening of blood vessels and other body lumens which are not suitable for stenting.

### BRIEF SUMMARY OF THE INVENTION

The present invention generally provides improved devices, systems, and methods for inducing desirable temperature effects on body tissue using non-RF energy. The desirable temperature effects include mildly heating the tissue for treating atherosclerotic lesions and other disease states. While also being well-suited for treatment of occlusive diseases, the techniques of the present invention are particularly advantageous for treatment of patients who have (or are at risk of having) vulnerable plaques, regardless of whether those vulnerable plaques cause significant occlusion of an associated vessel lumen. Catheter systems of the present invention can incorporate optical coherence tomography or other imaging techniques which allow a structure and location of the diseased tissue to be characterized.

In a first aspect, the invention comprises a system for inducing desirable temperature effects on body tissue, the body tissue being disposed about a lumen. The system includes an elongate catheter having a proximal end and a distal end with an axis therebetween with an energy delivery portion for transmission of energy. A tissue analyzer configured to characterize the body tissue in the lumen proximate the energy delivery portion and an energy source coupled to the energy delivery portion transmitting tissue treatment energy, wherein the energy is non-RF energy. A processor coupled to the tissue analyzer and energy source, the processor configured to determine an appropriate treatment energy for the characterized body tissue so as to mildly heat the body tissue with the energy delivery portion without ablating.

In another aspect, the invention comprises method for inducing desirable temperature effects on body tissue within a body lumen. The method includes positioning an energy delivery portion of a catheter within the lumen adjacent the tissue to be heated, characterizing the tissue in the lumen proximate the energy delivery portion using a tissue analyzer. Then determining an appropriate treatment energy for the characterized tissue using a processor coupled to the tissue analyzer and energizing the energy delivery portion with appropriate treatment energy from an energy source coupled to the processor. Mildly heating the tissue without ablating, with the appropriate treatment energy, without causing excessive thermal damage to the tissue so as to induce a long-term occlusive response.

In many embodiments the energy device comprises a laser energy source.

In many embodiments the energy delivery portion may comprises at least one radially oriented window coupled to at least one optical conduit extending between the proximal end of the catheter and the at least one window for transmission of laser energy to the body tissue from the laser energy source.

In many embodiments the energy device is an ultrasound energy source.

In many embodiments the energy delivery portion comprises at least one ultrasound transducer configured to deliver ultrasound energy to the body tissue. The frequency of the energy is between 150 kHz and 5 MHz.

In many embodiments the energy source is a microwave energy source.

In many embodiments the energy delivery portion comprises at least one microwave antenna configured to deliver microwave energy to the body tissue.

In many embodiments the processor has predetermined treatment energy characteristics suitable for mildly heating different characterized materials.

In many embodiments the processor is configured to adjust the treatment energy in response to feedback from the tissue analyzer during heating of the body tissue.

In many embodiments the tissue analyzer comprises an optical coherence tomographer coupled to at least one optical conduit extending between the proximal end of the catheter and at least one radially oriented window, the tomographer generating image signals from imaging light from the body tissue so as to characterize the body tissue, the imaging light transmitted through the at least one window.

In another aspect, the invention comprises a system for non-invasively inducing desirable temperature effects on tissue in a tissue treatment area within a body lumen. The system includes a focused ultrasound energy device configured to deliver focused ultrasound energy to the tissue treatment area, a tissue analyzer configured to characterize the tissue in the tissue treatment area, and a processor coupled to the tissue analyzer and focused ultrasound energy device, the processor configured to determine appropriate focused ultrasound parameters for the characterized tissue so as to mildly heat the tissue without ablating, with the appropriate focused ultrasound energy, without causing excessive thermal damage to the tissue so as to induce a long-term occlusive response.

In another aspect, the invention comprises a method for non-invasively inducing desirable temperature effects on tissue in a tissue treatment area within a body lumen. The method includes positioning a focused ultrasound energy device configured to deliver focused ultrasound energy to the tissue treatment area, characterizing the tissue in the lumen proximate the energy delivery portion using a tissue analyzer and determining an appropriate treatment energy for the characterized tissue using a processor coupled to the tissue analyzer and the focused ultrasound energy device. The focused ultrasound energy device is energized with appropriate treatment energy, mildly heating the tissue without ablating, with the appropriate treatment energy, without causing excessive thermal damage to the tissue so as to induce a long-term occlusive response.

### BRIEF DESCRIPTION OF THE DRAWINGS

FIG. 1 schematically illustrates one embodiment of a balloon catheter system for gently heating artery tissue.

FIG. 2 shows a plurality of ultrasonic transducers or microwave antennas mounted on a balloon surface for using in a catheter system.

FIG. 3 shows one embodiment of a plurality of transducers or antennas mounted on a stent-like cage for use with a catheter system.

FIG. 4 shows a cross-section view of a balloon and transducers inflated in an artery having plaque or lesion and calcium deposits.

FIG. 5 shows one embodiment of over-lapping wave patterns of ultrasonic energy when focusing transducers are used.

FIG. 6 shows a cross-section view of a balloon and transducers inflated in an artery with two different plaques being treated by the ultrasonic catheter.

FIG. 7 shows an alternative way to induce heat in an artery having plaque or lesion using an ultrasonic catheter that is depth/tissue specific for gentle heating.

FIG. 8 shows a pattern of waves emitted from unfocused single transducer.

FIG. 9 shows a cross-section view of a balloon with a single unfocused transducer inflated in an artery emitting unfocused ultrasonic energy into plaque or lesion for gentle heating.

FIGS. 10A-10C show cross-section views of a balloon inflated in an artery transmitting ultrasonic energy toward plaque or lesion using an array of transducers, a pair of transducers, or a single transducer.

FIG. 11 shows one embodiment of a catheter with transducers mounted on the inside surface of the balloon.

FIGS. 12A and 12B show one embodiment of non-invasive treatment using external ultrasound to treat a diseased or plaque portion of an artery within a body.

FIG. 13A shows an approximation of two point sources separated by a distance “s” having a wavelength less than “s”.

FIG. 13B shows an approximation of two point sources separated by a distance “s” having a wavelength greater than “s” for heating below a surface.

FIG. 14A shows an example of a minimum configuration with a spacing Sₘᵢₙ of 0.2 mm and depth dₘᵢₙ of 0.1 mm used in calculating a minimum frequency.

FIG. 14B shows an example of a maximum configuration with a spacing Sₘₐₓ of 2.0 mm and depth dₘₐₓ of 5.0 mm used in calculating a maximum frequency.

FIG. 15 shows one embodiment of laser based catheter system designed to gently heat body tissue using laser energy.

FIG. 16 shows a cross-section view of one embodiment of a distal end of the laser catheter of FIG. 15.

FIGS. 17A-17D show one embodiment of a laser catheter for gentle heating a body lumen.

### DETAILED DESCRIPTION OF THE INVENTION

Many therapies have been developed to replace or improve upon traditional balloon angioplasty and stents. The alternative devices described in the BACKGROUND OF THE INVENTION either cut, ablate, or vaporize diseased tissue in an artery. For example, laser devices vaporize plaque and flush it downstream. Atherectomy devices excise plaque and suck it out of the body. Cutting balloons incise the artery wall, damaging the tissue.

It would be advantageous to provide systems and devices that do not cut, ablate, or vaporize. Three modalities of treatment avoid these drawbacks and include: cooling the tissue; non-ablative forms of direct molecular denaturing; and non-ablative heating. Cooling has been implemented using devices such as Boston Scientific's Cryo-cath. Direct molecular denaturing can be achieved with radiation—gamma rays, for instance.

The present invention is directed to the remaining modality, non-ablative heating using non-RF energy. The embodiments disclosed herein revolve around the concept of “gentle heating” of diseased tissue in an artery, regardless of the specific treatment modality or technological implementation. In some embodiments, the treatment of the diseased artery is achieved using a device inside the artery without ablation, while other embodiments disclose non-invasive treatment from outside the body using external devices.

While generally described herein with reference to the vasculature, embodiments of the catheter devices, systems, and methods described herein may also find applications in the lumens of other vessels of the human anatomy. The anatomical structure into which the catheter is placed may be for example, the esophagus, the oral cavity, the nasopharyngeal cavity, the auditory tube and tympanic cavity, the sinus of the brain, the larynx, the trachea, the bronchus, the stomach, the duodenum, the ileum, the colon, the rectum, the bladder, the ureter, the ejaculatory duct, the vas deferens, the urethra, the uterine cavity, the vaginal canal, and the cervical canal, as well as the arterial system, the venous system, and/or the heart

Remodeling may involve the application of ultrasound energy, microwave energy, laser energy, and the like. This energy may be controlled so as to limit a temperature of target and/or collateral tissues, for example, limiting the heating of a fibrous cap of a vulnerable plaque or the intimal layer of an artery structure. In some embodiments, the surface temperature range for gentle heating is from about 45° C. to about 99° C. For mild gentle heating, the surface temperature may range from about 45° C. to about 65° C., while for more aggressive gentle heating, the surface temperature may range from about 65° C. to about 99° C. Limiting heating of a lipid-rich pool of a vulnerable plaque sufficiently to induce melting of the lipid pool while inhibiting heating of other tissues (such as an intimal layer or fibrous cap) to less than a surface temperature in a range from about 50° C. to about 65° C., such that the bulk tissue temperature remains mostly below 50° C.-55° C. may inhibit an immune response that might otherwise lead to restenosis, or the like. Relatively mild temperatures between 50° C. and 65° C. may be sufficient to denature and break protein bonds during treatment, immediately after treatment, and/or more than one hour, more than one day, more than one week, or even more than one month after the treatment through a healing response of the tissue to the treatment so as to provide a bigger vessel lumen and improved blood flow.

The length of time or average rate of energy delivery for gently heating of body tissue may also vary. For example, in some embodiments the average rate of energy delivery to the tissue is on the same order of magnitude as the rate of energy dissipation by the tissue. In other embodiments, the delivered energy is sufficiently low that differences in tissue properties, including thermal conduction, heat capacity, innate blood perfusion, and distance from well perfused tissue, cause heat to be drawn from the healthy tissue at a rate that avoids significant thermal damage to the healthy tissue, while allowing heat to build up in diseased tissue

The devices, systems and methods described herein are not selective in tissue treatment of the blood vessel and can be used for treatment of both concentric and eccentric atherosclerosis. This is a particular advantage because atherosclerosis may be eccentric relative to an axis of the blood vessel over 50% of the time, possibly in as much as (or even more than) 75% of cases.

Hence, remodeling of atherosclerotic materials may comprise shrinkage, melting, and the like of atherosclerotic and other plaques. Atherosclerotic material within the layers of an artery may be denatured, melted and/or the treatment may involve a shrinking of atherosclerotic materials within the artery layers so as to improve blood flow. The invention may also provide particular advantages for treatment of vulnerable plaques or blood vessels in which vulnerable plaque is a concern, which may comprise eccentric lesions. The invention will also find applications for mild heating of the cap structure (to induce toughening of the cap and make the plaque less vulnerable to rupture) and/or heating of the lipid-rich pool of the vulnerable plaque (so as to remodel, denature, melt, shrink, and/or redistribute the lipid-rich pool).

Catheter Based Treatment

Some embodiments of the present invention generally provide devices, systems, and methods for inducing desirable temperature effects on artery tissue, particularly atherosclerotic diseased tissue, by gentle heating in combination with gentle or standard dilation. In many embodiments the disclosed system consists of at least two elements, an energy generator and a catheter. The catheter may be similar to a balloon catheter commonly used to treat artery disease today, except for the addition of ultrasonic transducers or microwave antennas. The system will be able to treat diseased tissue by gentle heating in combination with dilation of the artery. In case of calcification in the artery plaque, it may be more difficult to remodel and open the diseased artery, so the catheter may use a standard angioplasty balloon in combination with ultrasonic energy to break down the calcium and remodel and open the lumen.

In one embodiment, an angioplasty balloon catheter structure having transducers disposed thereon might apply ultrasound heating to the vessel wall. In another embodiment, an angioplasty balloon catheter structure having microwave antennas disposed thereon might apply microwave heating to the vessel wall. The heating of the vessel wall may be done before, during, and/or after dilation, optionally in combination with dilation pressures which are at or significantly lower than standard, unheated angioplasty dilation pressures. For example, where balloon inflation pressures of 10-16 atmospheres may be appropriate for standard angioplasty dilation of a particular lesion, modified dilation treatments combined with gentle heating described herein may employ from 10-16 atmospheres or may be effected with pressures of 6 atmospheres or less, and possibly as low as 1 to 2 atmospheres.

The gentle heating energy added before, during, and or after dilation of a blood vessel may increase dilation effectiveness while lowering complications. In some embodiments, such controlled heating with balloon dilatation may exhibit a reduction in recoil, providing at least some of the benefits of a stent-like expansion without the disadvantages of an implant. Benefits of gentle heating may be enhanced (and/or complications inhibited) by limiting heating of the adventitial layer below a deleterious response threshold.

While the present invention may be used in combination with stenting, the present invention is particularly well suited for increasing the open diameter of blood vessels in which stenting is not a viable option. Potential applications include treatment of diffuse disease, in which atherosclerosis is spread along a significant length of an artery rather than being localized in one area. The invention may also find advantageous use for treatment of tortuous, sharply-curved vessels, as no stent need be advanced into or expanded within the sharp bends of many blood vessel. Still further advantageous applications include treatment along bifurcations (where side branch blockage may be an issue) and in the peripheral extremities such as the legs, feet, and arms (where crushing and/or stent fracture failure may be problematic).

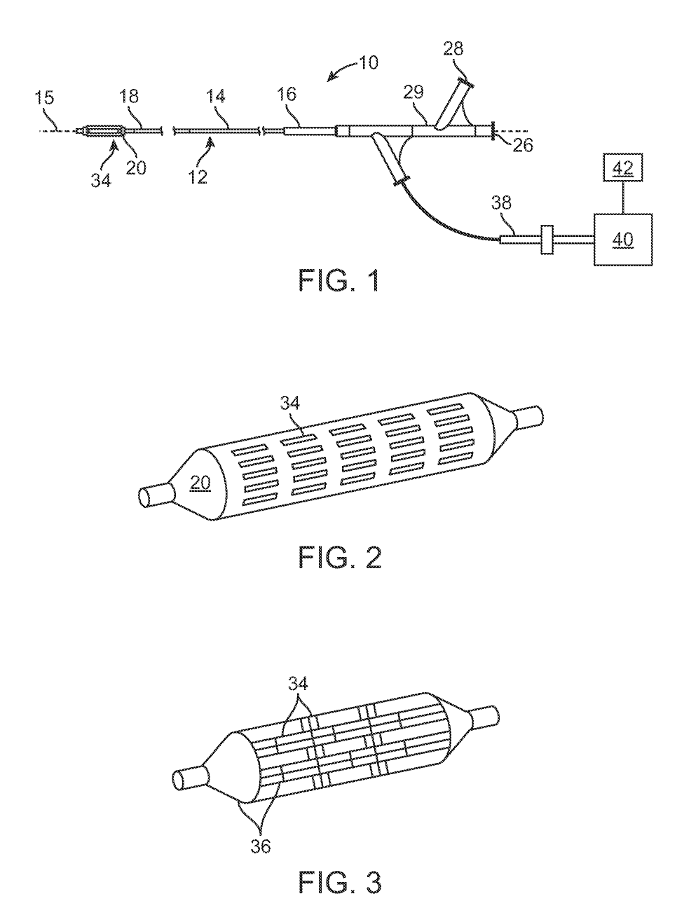

FIG. 1 shows one embodiment of a balloon catheter system 10 for inducing desirable temperature effects on artery tissue. The catheter system 10 includes a balloon catheter 12 having a catheter body 14 with a proximal end 16 and a distal end 18 with an axis 15 therebetween. Catheter body 14 is flexible and may include one or more lumens, such as a guidewire lumen and an inflation lumen. Still further lumens may be provided if desired for other treatments or applications, such as perfusion, fluid delivery, imaging, conductor lumen, or the like. Catheter 12 includes an inflatable balloon 20 adjacent distal end 18 and a housing 29 adjacent proximal end 16. Housing 29 includes a first connector 26 in communication with the guidewire lumen and a second connector 28 in fluid communication with the inflation lumen. The inflation lumen extends between balloon 20 and second connector 28. Both first and second connectors 26, 28 may optionally comprise a standard connector, such as a Luer-Loc™ connector. A distal tip may include an integral tip valve to allow passage of guidewires, and the like.

Ultrasound transducers or microwave antennas 34 are mounted on a surface of balloon 20, covering the balloon partially or fully, with associated conductors extending proximally from the transducers or antennas. In some embodiments, the transducers or antennas 34 may be positioned internal of balloon 20. Transducers or antennas 34 may be arranged in many different patterns or arrays on balloon 20. In some embodiments, adjacent transducers or antennas are axially offset. In other embodiments, transducers or antennas may be arranged in bands around the balloon. The transducers may also be focusing transducers.

Housing 29 also accommodates an electrical connector 38. Connector 38 includes a plurality of electrical connections, each electrically coupled to the transducers or antennas 34 on the balloon surface via conductors, shown in FIG. 2. This allows the transducers or antennas 34 to be easily energized by a controller 40 and power source 42, such as ultrasound energy, microwave energy, or other suitable energy sources.

In some embodiments, the balloon may be made as an ultrasonic transducer with a transmitting layer comprising piezoelectric material, having a relatively high dielectric constant and a relatively high acoustic impedance. Upon inflation, the balloon, which is actually the ultrasonic transducer, will emit ultrasonic energy toward the plaque. This system will be able to treat athrosclerosis disease by gentle heating with gentle or standard dilation.

The ultrasonic transducer balloon can be built in different forms, such as:
1. The entire balloon is made of piezoelectric plastic.
2. Only part of the balloon will be made of piezoelectric plastic, and thus can treat specifically diseased tissue, by rotating before inflation, and after deflation.
3. By using a receiving layer comprising piezoelectric material or any other suitable material, wherein the layers (transmitting and receiving) are interconnected in a laminar manner, the catheter will also be able to diagnose, e.g., IVUS catheter. This will enable detection of plaque depth and tissue type, during the treatment, using only one catheter.
4. Discrete subsets of the balloon surface material are embedded with ultrasonic transducer elements. Then, the discrete elements are independently connected to an external generator that can activate the elements independently and at independent power, frequency, etc., if desired. This independent activation allows for selective dosing within a lumen, as well as discretization of the treatment energy, which has other benefits. For instance, energy is more directed and evenly delivered rather than it flowing wherever the tissue composition and properties dictate, as is the case with non-selective, unfocused energy.

In other embodiments, the transducers 34 mentioned above can also be built in a form of a cage 36. For example, FIG. 3. shows an expandable cage built from transducers 34 mounted on a stent-like cage 36. This is possible with any method of creating apposition with the lumen wall. An advantage for a catheter built like this is that during treatment, the flow of fluid through the lumen doesn't stop. The stent-like cage with the transducers on it will be pulled out from the artery in the end of the treatment.

There is also the option in most embodiments of adding a transducer/antennas to the tip of the catheter in order to help with crossing a legion or blockage in the artery lumen.

Balloon 20 generally includes a proximal portion coupled to the inflation lumen and a distal portion coupled to the guidewire lumen. Balloon 20 expands radially when inflated with a fluid or a gas. In some embodiments, the fluid or gas may be non-conductive and/or cooled. In some embodiments, balloon 20 may be a low pressure balloon pressurized to contact the artery tissue. In other embodiments, balloon 20 is an angioplasty balloon capable of a higher pressure to expand the artery lumen while gentle heating is applied. Balloon 20 may comprise a compliant or non-compliant balloon having helical folds to facilitate reconfiguring the balloon from a radially expanded, inflated configuration to a low profile configuration, particularly for removal after use.

In some embodiments, controller 40 may include a processor or be coupled to a processor to control or record treatment. The processor will typically comprise computer hardware and/or software, often including one or more programmable processor unit running machine readable program instructions or code for implementing some or all of one or more of the methods described herein. The code will often be embodied in a tangible media such as a memory (optionally a read only memory, a random access memory, a non-volatile memory, or the like) and/or a recording media (such as a floppy disk, a hard drive, a CD, a DVD, a non-volatile solid-state memory card, or the like). The code and/or associated data and signals may also be transmitted to or from the processor via a network connection (such as a wireless network, an ethernet, an Internet, an intranet, or the like), and some or all of the code may also be transmitted between components of catheter system 10 and within processor via one or more bus, and appropriate standard or proprietary communications cards, connectors, cables, and the like will often be included in the processor. Processor will often be configured to perform the calculations and signal transmission steps described herein at least in part by programming the processor with the software code, which may be written as a single program, a series of separate subroutines or related programs, or the like. The processor may comprise standard or proprietary digital and/or analog signal processing hardware, software, and/or firmware, and will typically have sufficient processing power to perform the calculations described herein during treatment of the patient, the processor optionally comprising a personal computer, a notebook computer, a tablet computer, a proprietary processing unit, or a combination thereof. Standard or proprietary input devices (such as a mouse, keyboard, touchscreen, joystick, etc.) and output devices (such as a printer, speakers, display, etc.) associated with modern computer systems may also be included, and processors having a plurality of processing units (or even separate computers) may be employed in a wide range of centralized or distributed data processing architectures.

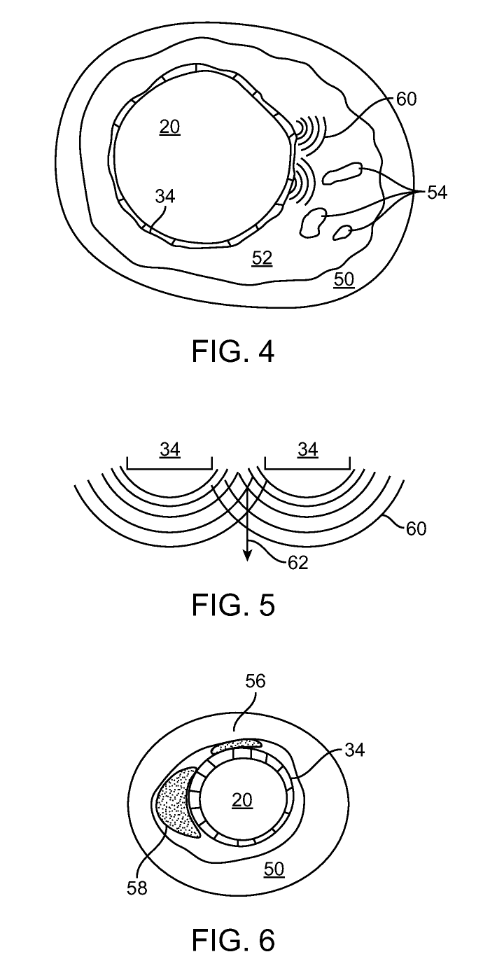

FIG. 4 shows a cross-section view of balloon 20 and transducers 34 inflated in an artery 50 having plaque or lesion 52 and calcium deposits 54. The transducers 34 emit ultrasonic energy 60 into a plaque or lesion 52. The ultrasonic energy 60 creates heat and mildly heats the plaque or lesion 52. The ultrasonic energy 60 may potentially break up the calcium deposits 54. The heat has numerous potential advantages, including collagen shrinkage and debulking Breaking calcium is also an advantage over existing products. Note also that the native vessel or artery 50 helps keep pressure on the plaque or lesion 52 to keep it in contact with transducers 34.

FIG. 5 shows one embodiment of over-lapping wave patterns 60 of ultrasonic energy when focusing transducers 34 are used. Arrow 62 represents the location of hotspots, which are locations where waves from different transducers combine, causing points with higher temperatures. The arrow 62 direction represents the depth of the plaque. This system will enable imposing constructive interference. Using different frequencies will enable the system to modulate wavelength, thus focusing on different depth of the plaque. Considering that the plaque is not homogenous, this system will be able to be “disease-area/location-specific” by modulating differences in wavelength between the transducers.

FIG. 6 shows a cross-section view of balloon 20 and transducers 34 inflated in an artery 50 with two different plaques 56, 58 being treated by the ultrasonic catheter 10. Balloon 20 is covered partially or fully by transducers 34. The little plaque 56 will be treated with shorter waves because of its shallow depth. The deeper plaque 58 will be treated with longer waves because of its deeper depth. As the ultrasonic power increases, the higher the temperature inside the plaque.

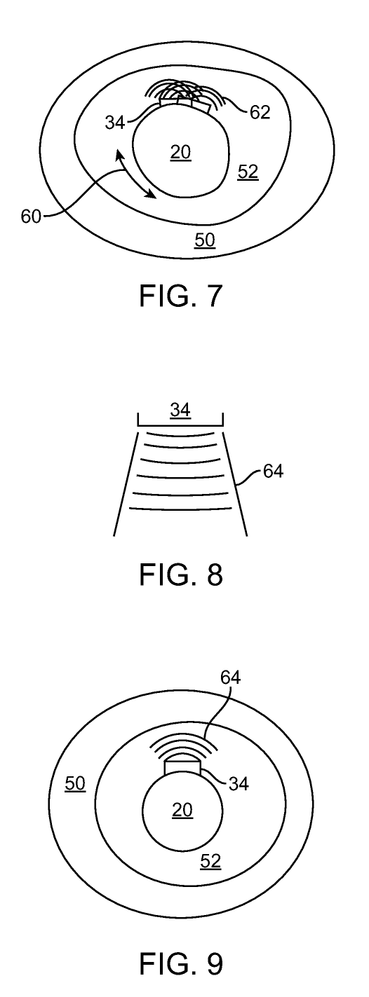

FIG. 7 shows an alternative way to induce heat in artery 50 having plaque or lesion 52 using an ultrasonic catheter that is depth/tissue specific for gentle heating. In this case, balloon 20 is only partially covered with focusing transducers 34, but the balloon is capable of rotating 60 within the artery, thus applying different wavelength 62 at a time to specific treatment areas, depending on the depth of the plaque, and the tissue type. This method will also avoid treating healthy tissue with no disease. This system can also be built with an unfocused single transducer.

FIG. 8 shows a pattern of waves 64 emitted from an unfocused single transducer 34. FIG. 9 shows a cross-section view of balloon 20 with single unfocused transducer 34 inflated in artery 50 emitting unfocused ultrasonic energy 64 into plaque or lesion 52 for gentle heating. Also in this embodiment, the power level may vary depending on the plaque depth and tissue type.

While the above embodiments disclose the transducers on the surface of the balloon, in other embodiments the location of the transducer(s) may be positioned inside the balloon. For example, a transducer wire/core may be positioned near a center of the balloon and be tuned to pass through saline and into tissue, preferably targeting disease or other unwanted components, e.g., calcium or thrombus.

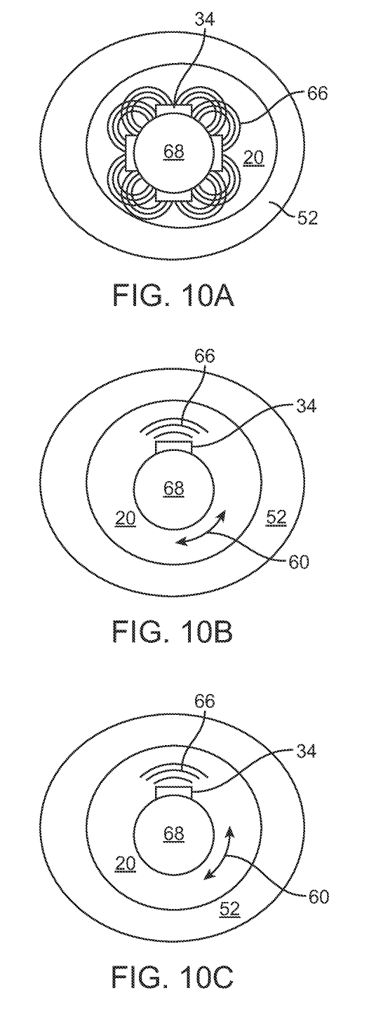

FIGS. 10A-10C show cross-section views of balloon 20 inflated in artery 50 transmitting ultrasonic energy 66 toward plaque or lesion 52. In the embodiment shown in FIG. 10A, a full array of focusing transducers 34 are on an inner core within balloon 20. In the embodiment shown in FIG. 10A, a pair of focusing transducers 34 are on an inner core 68 located within balloon 20 that can rotate 60. In the embodiment shown in FIG. 10A, an unfocused single transducer 34 is positioned on an inner core 68 within balloon 20 that can rotate 60.

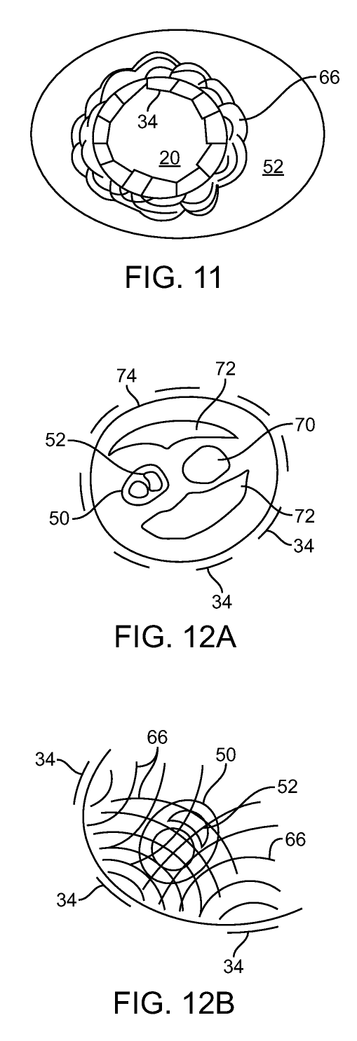

FIG. 11 shows one embodiment of a catheter with transducers 34 mounted on the inside surface of the balloon 20. In some embodiments, the balloon can act as a lens or diaphragm. The energy 66 in this case doesn't have to pass through saline, and there is less risk for delaminating transducers. This kind of catheter can also be built with one facet of focusing transducers on the inside surface of the balloon, that can rotate, after deflation. It also can be built with an unfocused single transducer on the inside surface of the balloon that can rotate.

The catheters disclosed above may be built using combinations of the embodiments, meaning that a catheter may be built with transducers on the outside surface of the balloon and also inside the balloon, whether in the center or inside surface of the balloon.

External Ultrasound

Another embodiment of the invention relates to systems and methods of non-invasively treating diseased or unwanted tissue or substance inside a human or animal body. In particular, this embodiment relates to using external, focused ultrasound, guided by imaging information used to treat atherosclerosis.

The basic principal of this embodiment is as follows: If you know where a plaque or diseased tissue is located and you know its topology (presumably from some kind of imaging device, such as MRI or IVUS, VH or otherwise), then the plaque or diseased tissue can be targeted using a focused ultrasound treatment that is external to the body.

FIGS. 12A and 12B show one embodiment using external ultrasound to treat a diseased or plaque portion 52 of an artery 50 within the body, for example, a leg 74 having muscles 72 and bone 70. A plurality of ultrasonic transducers 76 are positioned around the leg 74 transmitting ultrasonic energy 78 toward plaque or lesion 52. The ultrasonic energy 78 may potentially break up the calcium deposits. As the ultrasonic energy waves 78 from different transducers combine, points with higher temperatures are created to mildly heat the plaque or lesion 52.

If the location of a plaque or disease is known using an imaging device, a control system (processor) can be used to activate discretely and selectively the ultrasonic transducers with independently specified frequency, phase, power, etc., in order to target the diseased tissue as needed. This method has the advantage of being totally noninvasive and potentially a lot faster than traditional angioplasty.

Ultrasound Calculations Example

Calculations related to depth of penetration and minimum desired surface depth of treatment and its relationship to frequency are discussed below. Making some assumptions about how the ultrasonic transducers might be disposed about the treatment catheter, e.g., on a balloon, it is possible to estimate the appropriate wavelengths and therefore frequencies that would be useful in implementing an ultrasonic catheter with focused waves.

The waves must constructively interfere in order to be maximally effective (create the hot spots). Ultrasound sound travels at different speeds through different mediums. For these calculations, we will assume the sound travels through tissue at approximately the same speed through salt water, roughly 1500 m/s.

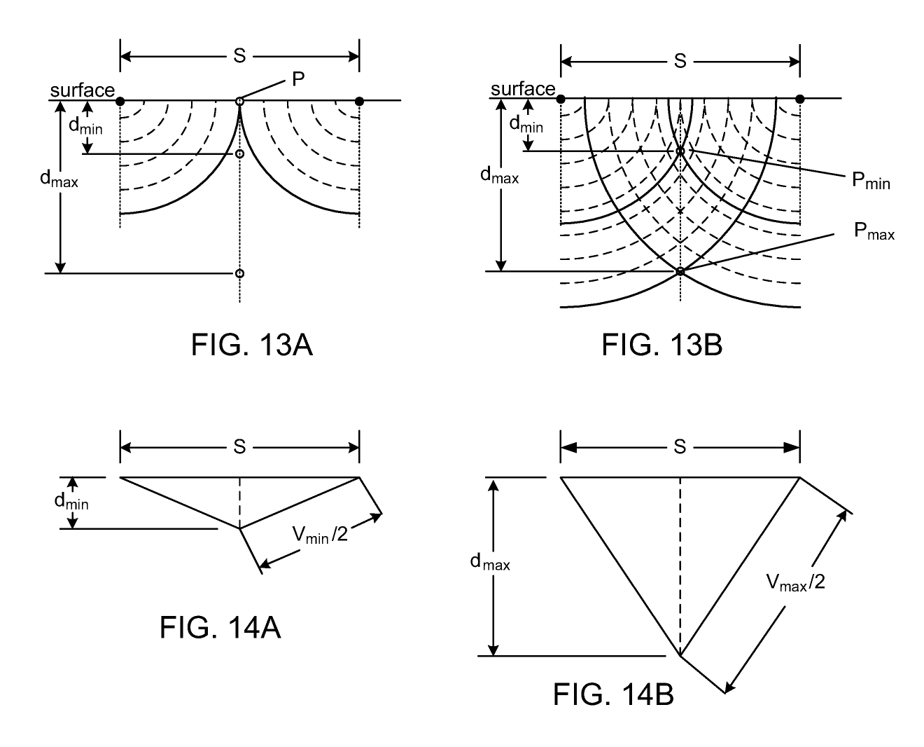

FIG. 13A shows an approximation of two point sources separated by a distance “s” having a wavelength less than “s”. This is not a particularly useful implementation because the focus of the energy and the maximum heating is at the surface between the sources where the waves intersect P. It is more useful to select a wavelength that allows one to create constructive interference below the surface of whatever organ or body lumen the transducers are there apposed. FIG. 13B shows the desired treatment for heating at a minimum depth dₘᵢₙ of 0.1 mm and a maximum depth dₘₐₓ of 5 mm into the lumen wall, with a spacing between sources of a minimum spacing of 0.2 mm to a maximum spacing of 2.0 mm. One set of range of wavelengths can be calculated using the formulas:

$$\begin{matrix}{f}_{min} = \frac{v}{{λ}_{min}} & ( a ) \\ λ = \frac{v}{f} & ( b )\end{matrix}$$

FIG. 14A shows a minimum configuration with a spacing Sₘᵢₙ of 0.2 mm and depth dₘᵢₙ of 0.1 mm, the wavelength λₘᵢₙ may be calculated as follows:

$$\begin{matrix}{( \frac{{λ}_{min}}{2} )}^{2} = {d}_{min}^{2} + {( \frac{{S}_{min}}{2} )}^{2} & ( c ) \\ {λ}_{min} = {2 [ {( 0.1 mm )}^{2} + {( \frac{0.2 mm}{2} )}^{2} ]}^{1 / 2} or 0.2828 mm & ( d )\end{matrix}$$

Solving now for maximum frequency,

$${f}_{max} = \frac{v}{{λ}_{min}} = \frac{1500 m \text{/} s}{0.2828 mm} = 5.3 MHz \qquad ( e )$$

FIG. 14B shows a maximum configuration with a spacing Sₘₐₓ of 2.0 mm and depth dₘₐₓ of 5.0 mm, the wavelength λₘₐₓ may be calculated as follows:

$$\begin{matrix}{( \frac{{λ}_{max}}{2} )}^{2} = {d}_{max}^{2} + {( \frac{{S}_{max}}{2} )}^{2} & ( f ) \\ {λ}_{max} = {2 [ {( 5.0 mm )}^{2} + {( \frac{2.0 mm}{2} )}^{2} ]}^{1 / 2} or 10.2 mm & ( g )\end{matrix}$$

Solving now for minimum frequency,

$${f}_{min} = \frac{v}{{λ}_{max}} = \frac{1500 m \text{/} s}{10.2 mm} = 147 kHz \qquad ( h )$$

The calculations suggest that an appropriate range of frequencies useful for this example of treating artery disease from transducers mounted on a balloon in the artery would be between roughly 150 kHz to 5 MHz. This frequency range is appropriate using a focused ultrasound approach, and more specifically a bipolar, two-transducer implementation. In other embodiments having target tissue at less depth or more depth, the frequency may range from 1 kHz to 20 MHz.

Laser Based Treatment

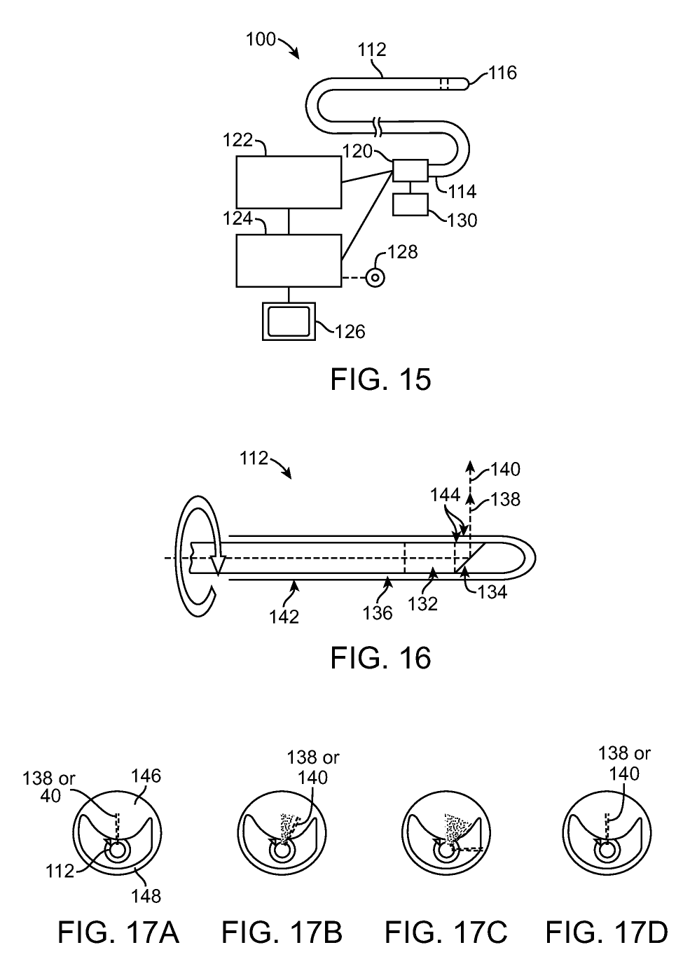

FIG. 15 shows one embodiment of a laser based catheter system 100 designed to gently heat body tissue using laser energy. A suitable system is disclosed in U.S. Patent Application No. 2005/0251116, filed May 3, 2005, entitled “Imaging And Eccentric Atherosclerotic Material Laser Remodeling And/Or Ablation Catheter”, the full disclosure of which is incorporated herein by reference.

The catheter system 100 includes a catheter 112 having a proximal end 114 and a distal end 116 with an axis therebetween. A housing 120 adjacent proximal end 114 couples the catheter to a heating laser 122 and an analyzer 124, the analyzer often comprising an optical coherence tomography system. Optionally, a display 126 may show intravascular optical coherence tomography (or other) images, and may be used by a surgeon in an image-guided procedure. A drive 130 may effect scanning for at least one imaging component relative to a surrounding catheter sleeve, the scanning optionally comprising rotational scanning, helical scanning, axial scanning, and/or the like.

Additional system components, such as an input device for identifying tissues on the display for treatment and a processor for interpreting the imaging light signals from catheter 112 will often be incorporated into a laser or imaging system, or may be provided as stand-alone components. Analyzer 124 will optionally include hardware and/or software for controlling laser 122, drive 130, display 126, and/or the like. A wide variety of data processing and control architectures may be implemented, with housing 120, drive 130, laser 122, analyzer 124 and or display 126 optionally being integrated into one or more structures, separated into a number different housings, or the like. Machine readable code with programming instructions for implementing some or all of the method steps described herein may be embodied in a tangible media 128, which may comprise a magnetic recording media, optical recording media, a memory such as a random access memory, read-only memory, or non-volatile memory, or the like. Alternatively, such code may be transmitted over a communication link such as an ethernet, Internet, wireless network, or the like.

Catheter 112 gently heats body tissue using laser energy in any of a variety of wavelengths, often ranging from ultraviolet to infrared. This energy may be delivered from laser 122 to a plaque or lesion by a fiber optic light conduit of catheter 112. Laser 122 may comprise an excimer laser using ultraviolet light or optionally use electrically excited xenon and chloride gases. Laser 122 may be either a continuous wave or pulsed laser. Continuous wave lasers often lead to deep thermal penetration and may lead to possible charring and shallow craters depending on the energy. A pulsed laser may reduce inadvertent heat conduction to surrounding tissues by providing sufficient time to permit thermal relaxation between pulses.

Referring now to FIGS. 16 and 17A-17D, catheter 112 generally uses one or more bundles of one or more rotatable optical conduits (sometimes referred to as “optical probes”) to direct light energy towards an artery wall at a given angle. The optical conduits may comprise one or more single-mode optical fiber, and may be housed inside a sleeve catheter or guidewire. The optical conduits may, at least in part, define optical paths, and each optical path may also be defined by a lens 132, and a fold mirror 134. The optical conduits may be used to convey light energy. The same optical conduit or bundle of optical conduits 136 may be used to convey the light energy for imaging, say imaging light 138, and the light energy for heating atherosclerotic plaques, say remodeling light 140. The optical conduits 136 are housed inside a sleeve catheter or guidewire 142.

The optical conduits 136 rotate inside the sleeve catheter 142. The imaging light 138 runs through the optical conduits and radially through transparent cylindrical windows 142 to provide an intra-vascular image of the body lumen, for instance by OCT. The image is processed by a computer that identifies and localizes atherosclerotic plaques. Based on the information from the imaging, the computer then determines when to fire the heating light 140 such that the light gently heats the plaque 146 and does not damage the healthy area 148 of the artery. This may be done by pulsing light 140 on the plaque when the rotatable optical conduits face the plaque. The imaging and heating lights may be used sequentially.

Embodiments of the devices, system, and methods described herein may adjusted or tuned the energy to gently heat the atherosclerotic materials. Characteristics of the energy, including the frequency, power, magnitude, delivery time, delivery location, and/or patterns or combinations thereof may be predetermined before diagnosis or treatment of a specific patient, the energy characteristics being transmitted without feedback, such as by employing open-loop dosimetry techniques. Such predetermined characteristic tuning may be based on prior treatment of atherosclerotic materials, prior clinical trials, and/or other development work. Some embodiments may tune the energy directed to a particular patient based on in situ feedback, and many embodiments may employ some predetermined characteristics with others being feedback-controlled.

While the exemplary embodiments have been described in some detail, by way of example and for clarity of understanding, those of skill in the art will recognize that a variety of modifications, adaptations, and changes may be employed. Hence, the scope of the present invention should be limited solely by the claims.

What is claimed is:

1. A system for inducing desirable temperature effects on body tissue, the body tissue being disposed about a lumen, the system comprising:
an elongate catheter having a proximal end and a distal end with an axis therebetween, the catheter having an energy delivery portion for transmission of energy;
a tissue analyzer configured to characterize the body tissue in the lumen proximate the energy delivery portion;
an energy source coupled to the energy delivery portion transmitting tissue treatment energy, wherein the energy is non-RF energy; and
a processor coupled to the tissue analyzer and energy source, the processor configured to determine an appropriate treatment energy for the characterized body tissue so as to mildly heat the body tissue with the energy delivery portion without ablating.

2. The system of claim 1, wherein the energy source comprises a laser energy source.

3. The system of claim 2, wherein the energy delivery portion comprises at least one radially oriented window coupled to at least one optical conduit extending between the proximal end of the catheter and the at least one window for transmission of laser energy to the body tissue from the laser energy source.

4. The system of claim 1, wherein the energy source is an ultrasound energy source.

5. The system of claim 4, wherein the energy delivery portion comprises at least one ultrasound transducer configured to deliver ultrasound energy to the body tissue.

6. The system of claim 4, wherein the frequency of the energy is between 1 kHz to 20 MHz.

7. The system of claim 1, wherein the energy source is a microwave energy source.

8. The system of claim 7, wherein the energy delivery portion comprises at least one microwave antenna configured to deliver microwave energy to the body tissue.

9. The system of claim 1, wherein the processor has predetermined treatment energy characteristics suitable for mildly heating different characterized materials.

10. The system of claim 1, wherein the processor is configured to adjust the treatment energy in response to feedback from the tissue analyzer during heating of the body tissue.

11. The system of claim 1, wherein the tissue analyzer comprises an optical coherence tomographer coupled to at least one optical conduit extending between the proximal end of the catheter and at least one radially oriented window, the tomographer generating image signals from imaging light from the body tissue so as to characterize the body tissue, the imaging light transmitted through the at least one window.

12. A method for inducing desirable temperature effects on body tissue within a body lumen, the method comprising:
positioning an energy delivery portion of a catheter within the lumen adjacent the tissue to be heated;
characterizing the tissue in the lumen proximate the energy delivery portion using a tissue analyzer;
determining an appropriate treatment energy for the characterized tissue using a processor coupled to the tissue analyzer;
energizing the energy delivery portion with appropriate treatment energy from an energy source coupled to the processor, wherein the energy is non-RF energy; and
gently heating the tissue without ablating, with the appropriate treatment energy, and without inducing a long-term occlusive response caused by excessive thermal damage to the tissue.

13. The method of claim 12, wherein the energy source comprises a laser energy source.

14. The method of claim 13, wherein the energy delivery portion comprises at least one radially oriented window coupled to at least one optical conduit extending between the proximal end of the catheter and the at least one window for transmission of laser energy to the tissue from the laser energy source.

15. The method of claim 12, wherein the energy source comprises an ultrasound energy source.

16. The method of claim 15, wherein energy delivery portion comprises at least one ultrasound transducer configured to deliver ultrasound energy to the body tissue.

17. The method of claim 15, wherein the frequency of the energy is between 1 kHz to 20 MHz.

18. The method of claim 12, wherein the energy source comprises a microwave energy source.

19. The method of claim 18, wherein the energy delivery portion comprises at least one microwave antenna configured to deliver microwave energy to the body tissue.

20. The method of claim 12, wherein the heating of the tissue is between 45 and about 99° C.

21. The method of claim 12, wherein the average rate of energy delivery to the tissue is on the same order of magnitude as the rate of energy dissipation by the tissue.

22. A system for inducing desirable temperature effects on body tissue having both healthy tissue and diseased tissue, the body tissue being disposed about a lumen, the system comprising:
an elongate catheter having an energy delivery portion for transmission of energy;
an energy source coupled to the energy delivery portion transmitting tissue treatment energy, wherein the energy is non-RF energy; and
a processor coupled to the tissue analyzer and energy source, the processor configured to determine an appropriate treatment energy to mildly heat the body tissue without ablating, to a temperature sufficient to efficaciously alter the diseased tissue, without causing excessive thermal damage to the healthy tissue so as to induce a long-term occlusive response.

23. The system of claim 22, wherein the energy source comprises a laser energy source and the energy delivery portion comprises at least one radially oriented window coupled to at least one optical conduit extending between the proximal end of the catheter and the at least one window for transmission of laser energy to the tissue treatment area from the laser energy source.

24. The system of claim 22, wherein the energy source is an ultrasound energy source and the energy delivery portion comprises at least one ultrasound transducer configured to deliver ultrasound energy to the tissue treatment area.

25. The system of claim 22, wherein the energy source is a microwave energy source and the energy delivery portion comprises a plurality of microwave antennas configured to deliver microwave energy to the tissue treatment area.

26. A system for non-invasively inducing desirable temperature effects on tissue in a tissue treatment area within a body lumen, the system comprising:
a focused ultrasound energy device configured to deliver focused ultrasound energy to the tissue treatment area;
a tissue analyzer configured to characterize the tissue in the tissue treatment area; and
a processor coupled to the tissue analyzer and focused ultrasound energy device, the processor configured to determine appropriate focused ultrasound parameters for the characterized tissue so as to gently heat the tissue with the appropriate focused ultrasound energy, without ablating, and without inducing a long-term occlusive response caused by excessive thermal damage to the tissue.

27. The system of claim 26, wherein the average rate of focused ultrasound energy delivery to the tissue treatment area is on the same order of magnitude as the rate of energy dissipation by the tissue treatment area.

28. The system of claim 26, wherein the tissue treatment area includes both healthy tissue and diseased tissue and the appropriate focused ultrasound energy will heat the diseased to a temperature sufficient to efficaciously alter the diseased tissue without causing excessive thermal damage to the healthy tissue.

29. The system of claim 26, wherein the tissue treatment area includes both healthy tissue and diseased tissue and the appropriate focused ultrasound energy is sufficiently low that differences in tissue properties, including thermal conduction, heat capacity, innate blood perfusion, and distance from well perfused tissue, cause heat to be drawn from the healthy tissue at a rate that avoids significant thermal damage to the healthy tissue, while allowing heat to build up in diseased tissue.

30. The system of claim 26, wherein the tissue analyzer is a magnetic resonance imaging (MRI) device.

31. The system of claim 26, wherein the processor is configured to adjust the focused ultrasound energy in response to feedback from the tissue analyzer during heating of the tissue.

32. The system of claim 26, wherein the processor has predetermined focused ultrasound energy characteristics suitable for gently heating different characterized tissues.

33. A method for non-invasively inducing desirable temperature effects on tissue in a tissue treatment area within a body lumen, the system comprising:
positioning a focused ultrasound energy device configured to deliver focused ultrasound energy to the tissue treatment area;
characterizing the tissue in the lumen proximate the energy delivery portion using a tissue analyzer;
determining an appropriate treatment energy for the characterized tissue using a processor coupled to the tissue analyzer and the focused ultrasound energy device;
energizing the focused ultrasound energy device with appropriate treatment energy; and
gently heating the tissue with the appropriate treatment energy, without ablating, and without inducing a long-term occlusive response caused by excessive thermal damage to the tissue.

34. The method of claim 33, wherein the average rate of focused ultrasound energy delivery to the tissue treatment area is on the same order of magnitude as the rate of energy dissipation by the tissue treatment area.

35. The method of claim 33, wherein the tissue treatment area includes both healthy tissue and diseased tissue and the appropriate focused ultrasound energy will heat the diseased tissue to a temperature sufficient to efficaciously alter the diseased tissue without causing excessive thermal damage to the healthy tissue.

36. The method of claim 33, wherein the tissue treatment area includes both healthy tissue and diseased tissue and the appropriate focused ultrasound energy is sufficiently low that differences in tissue properties, including thermal conduction, heat capacity, innate blood perfusion, and distance from well perfused tissue, cause heat to be drawn from the healthy tissue at a rate that avoids significant thermal damage to the healthy tissue, while allowing heat to build up in diseased tissue.

37. The method of claim 33, wherein the tissue analyzer is a magnetic resonance imaging (MRI) device.

38. The method of claim 33, wherein the processor is configured to adjust the focused ultrasound energy in response to feedback from the tissue analyzer during heating of the tissue.

39. The method of claim 33, wherein the processor has predetermined focused ultrasound energy characteristics suitable for mildly heating different characterized tissues.
